(19) United States
(12) Patent Application Publication
(10) Pub. No.: US 2023/0068571 A1
(43) Pub. Date: Mar. 2, 2023

(54) SYSTEM AND METHOD OF MANAGING WORKFLOW OF EXAMINATION OF PATHOLOGY SLIDES

(71) Applicant: IBEX MEDICAL ANALYTICS LTD., Tel Aviv (IL)

(72) Inventors: Chaim LINHART, Hod Hasharon (IL); Issar YAZBIN, Rehovot (IL); Gev DECKTOR, Tel Aviv (IL); Lilach BIEN, Rehovot (IL); Roei HARDUF, Ramat-Gan (IL); Cobi REOUVEN, Givatayim (IL)

(73) Assignee: IBEX MEDICAL ANALYTICS LTD., Tel Aviv (IL)

(21) Appl. No.: 17/799,925

(22) PCT Filed: Dec. 22, 2020
(86) PCT No.: PCT/IL2020/051319

§ 371 Date: Aug. 16, 2022

Related U.S. Application Data
(60) Provisional application No. 62/981,925, filed on Feb. 26, 2020.

(51) Int. Cl. G02B 21/36; G06T 7/00

(52) U.S. Cl. CPC G02B 21/367; G06T 7/0012; G06T 2207/20081

(57) ABSTRACT

A method and a system for managing a process of pathology examination may include an image-analysis module, configured to receive at least one digital scan of at least one pathology slide related to a pathology case, and perform at least one algorithm of image analysis thereon to extract at least one clinical feature of the at least one slide; a human-machine interface, adapted to present the at least one digital scan on a screen; a machine-learning based module, adapted to classify the at least one pathology slide to a classification of a set of classifications based on at least one clinical feature, the set of classifications selected from a list consisting a low risk classification and a high risk classification; and a report module, adapted to produce, in real time, at least one report data element comprising diagnostic information, based on the classification of the at least one pathology slide.

### FIELD OF THE INVENTION

The present invention relates generally to digital pathology. More specifically, the present invention relates to managing workflow of examination of pathology slides.

### BACKGROUND OF THE INVENTION

Recent years have seen the rise of digital pathology, also referred to as virtual microscopy—the practice of pathology using scanned or imaged slides on a computer screen, rather than analyzing glass slides through a conventional microscope. This new modality has been driven by technological advancements in areas such as digital storage capacity, network speed and computer processing power. Whole slide image (WSI) scanners are used in many institutes around the world for training pathologists, research, tele-pathology, consultation, archiving and, most recently, routine clinical diagnosis.

Digitization of pathology slides has also allowed the introduction of new technologies that facilitate the pathologist's work, e.g., workflow software, measurement, and annotation utilities and image sharing.

Computational Pathology, i.e., the automatic analysis of pathology images using advanced algorithms, has the potential to completely revolutionize the practice of pathology. Extant applications include automatic and semi-automatic quantification of immunohistochemistry (IHC) slides, mitosis counting, slide registration (e.g., aligning Hematoxylin and Eosin (H&E) slides with corresponding IHC slides), and the like.

Various problems in computational pathology have been studied in the academic literature, including for example: image registration, 3D reconstruction, stain separation and standardization, feature extraction (e.g., glands, mitoses, and the like), cancer detection and cancer grading.

Commercially available software tools provide analysis of WSIs for both clinical diagnosis and research applications, although most existent clinical applications exhibit a limited scope, in the sense that they do not attempt to perform a full diagnosis. Instead, they produce a localized analysis, in tasks such as IHC quantification, tissue segmentation and image registration.

Together with the growing adoption of digital pathology and automated diagnostic tools, pathology institutes face new challenges, namely, how to utilize the benefits of novel machine-learning based tools, to optimize throughput, decrease turnaround time (TAT), increase accuracy and improve consistency and quality of reports of pathology institutes and/or individual pathologists.

### SUMMARY OF THE INVENTION

It may be appreciated by a person skilled in the art that currently available systems for analysis of digitally scanned pathology slides do not include comprehensive integration of the analysis of WSIs with a workflow of a pathology specialist in a pathology lab or institute. The term “comprehensive” may, in this context, refer to a level of integration that is beyond that of currently available technology of Image Analysis (IA)-based processes of pathology slide examination, as demonstrated by the examples that follow.

It should be emphasized that the long felt need for integration of automated WSI analysis with workflow of pathology experts has so far resulted in a sub-optimal workflow of the examination process. In fact, field trials have shown that sub-optimal integration of the IA process into the workflow of pathology examination has led to slow adoption rate of digital pathology and even to erroneous, or life threatening consequences.

It may be appreciated by a person skilled in the art that currently available systems may not include a common platform that may integrate (a) a pathology analysis process performed by an automated IA module; (b) a process of examination performed by a human pathology expert and (c) a process of producing a pathology report. As elaborated herein, some embodiments of the invention may perform such required integration, to obtain synergy among the integrated elements (a), (b) and (c) in order to improve each of the integrated elements and to produce a comprehensive workflow for examination of a pathology case in a pathology laboratory.

For example: some embodiments of the invention may integrate (a) into (b) in order to facilitate triage of a pathology case and/or routing the pathology case to a specific human pathology expert (e.g., based on the pathologist's workload, expertise, experience, error rate, etc.). Additionally, or alternatively, some embodiments of the invention may include integration of information from external data sources in order to further improve such triage. For example, some embodiments of the invention may include integration of a Lab Information System (LIS) and perform triage based on the (a) and further based on information from the LIS (e.g., information pertaining to a pathologists' workload, expertise, etc.).

In another example, as elaborated herein, integration of (a) into (b) may enable some embodiments of the invention to produce a workflow in which different pathology cases and/or digitally scanned pathology slides may undergo a respective different process of preparation and/or examination, based on pathological findings (e.g., levels of severity, determined by the automated IA process) of each case and/or slide.

In another example, integration of (a) into (b) may enable some embodiments of the invention to produce a workflow, according to which a pathology expert may be presented or guided through regions of interest (ROIs) of a specific pathology case (e.g., based on suspected severity of each ROI).

Moreover, integration of (a) into (b) may provide a mechanism for controlling the process of examination, that may include a check-list, to ensure a thorough, orderly examination by the human pathology expert, and to prevent a condition in which an important ROI is missed.

In another example, integration of (b) into (a) may, for example facilitate improvement of the IA process through supervised training or labeling, provided by the human expert.

In another example, integration of (a) into (c) may, for example, facilitate automated production of at least a portion of a pathology report that may include at least one finding provided by the automated IA process. Such automated production of at least a portion of a pathology report may save effort and time of a human pathology expert, as well as errors in manual reporting.

In another example, integration of (a) into (c) may provide a mechanism of quality control (QC) over the report to prevent erroneous or lacking report by the human pathology expert. For example, some embodiments of the invention may be configured to find discrepancies between a content of a pathology report (e.g., having no medical findings) and IA findings (e.g., ROIs suspected as cancerous regions).

In another example, integration of (a) into (c) may enable some embodiments of the invention to find discrepancies between a content of a pathology report and content of additional data sources (e.g., a Lab Information System) associated with the pathology case (e.g., a reported mammography scan that may include regions of calcification).

In another example, integration of (a) and (b) into (c) may enable some embodiments of the invention to digitally associate specific findings of a pathology examination with specific locations or ROIs in a digitally scanned pathology slide for future or retroactive examination of the pathology case. For example, some embodiments of the invention may enable a user to follow through a previously conducted examination process (e.g., in retrospect) and determine whether specific locations or ROIs pertaining to the pathology case have been properly viewed (e.g., using a proper magnification or zoom level) and/or properly diagnosed (e.g., in a condition that a diagnosis by a human pathologist does not comply with findings of the pathology analysis process of the automated IA module (a)).

In another example, integration of (c) into (a) and (b) may enable some embodiments of the invention to interactively present, on a screen, elements of the pathology report in conjunction with respective ROIs in a digitally scanned pathology slide. For example, some embodiments may enable a pathology expert to select findings or elements included in the report and thus navigate among and/or highlight respective findings or ROIs in the presented image of the digitally scanned pathology slide. Additionally, or alternatively, some embodiments may enable a pathology expert to select specific ROIs on a presented image of a digitally scanned pathology slide and thus navigate to a corresponding region of the pathology report, and/or create a new portion of the report, corresponding to the selected ROI.

It may be appreciated by a person skilled in the art that currently available systems may not provide morphologic and/or graphical properties of elements in digitally scanned pathology slides, including for example, a number of examined tissue fragments, a length of one or more tissue fragments, a property of a shape of one or more tissue fragment, a dimension (e.g., a length, a volume, etc.) of a tumor (e.g., across one or more digitally scanned pathology slides), a tumor percentage (e.g., a number or percentage of cancerous cells or cancerous ROIs within an overall tissue area on one or more digitally scanned pathology slides), a percentage of different grades of ROIs (e.g., gradings of cells) within a tumor and/or within a slide, etc.

Some embodiments of the invention may include analysis of such morphologic and/or graphical properties. Furthermore, integration of (a) into (c) may enable some embodiments of the invention to include an outcome of analysis of morphologic and/or graphical properties in the process of pathology examination and/or reporting.

Moreover, some embodiments of the invention may cross-correlate an outcome of the analysis of morphologic and/or graphical properties with data and/or metadata (e.g., from an LIS system) pertaining to the pathology slides. For example, some embodiments of the invention may identify a condition of discrepancy between a morphologic and/or graphical property (e.g., a number and/or length of tissue fragments) as reported during preparation of the slides and/or by a physician who extracted the respective pathology sample or biopsy, and a corresponding property (e.g., number of fragments) that have actually been received by and/or examined by the pathologist.

In view of the non-exhaustive list of examples brought above, it may be appreciated by a person skilled in the art that some embodiments of the invention may provide a solution to a long-lasting need for integration of separate elements of a pathology examination process, so as to produce and/or manage a comprehensive workflow of pathology examination in a pathology institute or laboratory.

Embodiments of the present invention may include a system for managing a process of pathology examination. Embodiments may include, for example, an IA module, configured to receive at least one digital scan of at least one pathology slide related to a pathology case, and perform at least one algorithm of image analysis thereon to extract at least one clinical feature of the at least one slide; a human-machine interface (HMI), adapted to present the at least one digital scan on a screen; a first machine-learning (ML) based module, adapted to classify the at least one pathology slide to a classification of a set of classifications based on at least one clinical feature, the set of classifications selected from a list consisting a low risk classification and a high risk classification; and a report module, adapted to produce in real time, at least one report data element comprising diagnostic information, based on the classification of the at least one pathology slide.

According to some embodiments of the invention, the at least one extracted clinical feature may correspond to one or more ROIs of the at least one slide, and the first ML-based module may be adapted to classify the ROIs to a classification of the set of classifications based on the at least one extracted clinical feature. According to some embodiments of the invention, classifying the at least one pathology slide may be done based on classification of the one or more ROIs of the at least one pathology slide.

According to some embodiments of the invention, the first ML-based module may be adapted to classify pathology case to a classification of the set of classifications, based on the at least one at least one classifications of a pathology slide related to the pathology case.

According to some embodiments of the invention, the HMI may be configured to receive, from a user, at least one viewing context data element, and present the digitally scanned pathology slide according to the at least one viewing context data element, in conjunction with the at least one diagnostic report data element on the screen. The at least one viewing context data element may be, or may include, for example, a selection of a pathology case, a selection of a specific digitally scanned pathology slide, a selection of one or more ROIs, a selection of panning, a selection of zoom, a selection of brightness and a selection of contrast.

According to some embodiments of the invention, the HMI may be configured to produce a viewing suggestion according to at least one of: the at least one extracted clinical feature, a classification of at least one ROI and a classification of at least one pathology slide.

According to some embodiments of the invention, the HMI may be configured to receive (e.g., from a user) at least one expert data element pertaining to the presented at least one pathology slide. The report module may be configured to integrate the at least one expert data element into the at least one report data element to produce an integrated report data element. Additionally, the HMI may be configured to present the integrated report data element on the screen according to the at least one viewing context data element.

Embodiments may include a quality control module. According to some embodiments of the invention, the quality control module may be configured to receive (e.g., from an LIS) at least one data element (e.g., an LIS data element) pertaining to the pathology case. the quality control module may analyze the integrated report data element in view of the at least one data element (e.g., the LIS data element) to detect at least one discrepancy. Additionally, the HMI may be configured to present a notification of at least one detected discrepancy on the screen.

Embodiments may include an ML-based optimization module, configured to: receive at least one digital scan of a pathology slide of the low risk classification; select a number N_{(POI)} of ROIs of low risk classification, of the least one received digitally scanned pathology slide; and produce a recommendation to examine the N_{(POI)} ROIs of the least one received digitally scanned pathology slide. According to some embodiments of the invention, the ML-based optimization module may be trained to select a minimal number N_{(POI)} of ROIs, so as to optimize a throughput of the process of pathology examination, while maintaining a predefined level of accuracy of the process of pathology examination.

Embodiments of the invention may include a workflow manager module, adapted to: produce an ordered list of examination of the plurality of digitally scanned pathology slides based on one or more ordering criteria; and present at least one digitally scanned pathology slide for examination via the HMI based on the ordered list of examination. The one or more ordering criteria may be, or may include, for example a classification of at least one pathology case, a classification of at least one scanned pathology slide, a classification of at least one ROI, at least one extracted clinical feature, a profile of at least one expert pathologist, and at least one LIS data element pertaining to the pathology case.

According to some embodiments of the invention, the set of classifications further may include a borderline risk classification.

Embodiments of the invention may include an ML-based preordering module, configured to: receive at least one of a clinical feature and a classification of a digitally scanned pathology slide; and produce at least one recommendation for preparation of a number N(SLIDES) of digitally scanned pathology slides. According to some embodiments of the invention, the ML-based preordering module may be trained to produce a minimal number N(SLIDES) of slides, so as to optimize a throughput of the process of pathology examination while maintaining a predefined level of accuracy.

According to some embodiments of the invention, the IA module may be configured to extract at least one tissue-related property pertaining to one or more tissue fragments of the at least one pathology slide. The tissue-related property may be or may include, for example a length of a tissue fragment, a shape of a tissue fragment, an area of a tissue fragment, a volume of a fragment and a number of tissue fragments in the scanned pathology slide.

Embodiments of the invention may include a quality control module, configured to analyze at least one tissue-related property of a tissue fragment pertaining to a specific pathology case, so as to determine a discrepancy between metadata of one or more scanned pathology slides of the specific pathology case, and the tissue-related property.

According to some embodiments of the invention, the report module may be further configured to integrate at least one tissue-related property of a tissue fragment, pertaining to a specific pathology case, into the at least one report data element, to produce an integrated report data element.

According to some embodiments of the invention, the at least one clinical feature may be or may include, for example, a high priority medical condition, a secondary medical condition, one or more ROIs corresponding to the high priority medical condition, one or more ROIs corresponding to the secondary medical condition, a diagnosis of the high priority medical condition, a diagnosis of the secondary medical condition, a grade of the high priority medical condition, a stage of the high priority medical condition, a morphologic property of the high priority medical condition, a morphologic property of the secondary medical condition, a cytologic property of the high priority medical condition and a cytologic property of the secondary medical condition.

According to some embodiments of the invention, the IA module may be configured to extract at least one slide-related property pertaining to the scanned pathology slide, and wherein the slide-related property may be or may include, for example, a location of an extracted clinical feature within the slide, a quantization of the extracted clinical feature within the slide, a dimension of a tumor across one or more digitally scanned pathology slides, a percentage of a tumor across one or more digitally scanned pathology slides, a mitoses count, a percentage per grade, an IHC quantification, an indication of a distance between cancerous cells and a border of a tissue, and an indication of whether cancerous cells have metastasized beyond the predefined border.

According to some embodiments of the invention, the report module may be configured to integrate at least one slide-related property of a slide pertaining to a specific pathology case into the at least one report data element, to produce an integrated report data element.

According to some embodiments of the invention, the IA module may be configured to: receive one or more data elements selected from a list consisting: (a) an expert pathologist data element pertaining to diagnosis of a presented pathology slide and (b) a pathology report data element; and use the received one or more data elements as supervisory data, to further train and improve the extraction of the at least one clinical feature.

Embodiments of the invention may include a method of managing a process of pathology examination Embodiments may include, for example, receiving at least one digital scan of at least one pathology slide related to a pathology case, and performing at least one algorithm of image analysis thereon to extract at least one clinical feature of the at least one slide; classifying at least one ROI of the at least one digital scan of at least one pathology slide to a classification of a set of classifications, based on at least one clinical feature; producing a workflow data element based on the classification of at least one ROI; and controlling the process of diagnosis, based on the at least one workflow data element, by guiding a pathologist through the diagnostic process on a human-machine interface.

Embodiments of the invention may include a method of managing a process of pathology examination. Embodiments may include, for example, receiving, by an IA module, at least one digital scan of at least one pathology slide related to a pathology case; performing, by the IA module, at least one algorithm of image analysis on the at least one digital scan, to extract at least one clinical feature of the at least one slide; classifying, by an ML based module, the at least one pathology slide to a classification of a set of classifications, based on at least one clinical feature, the set of classifications selected from a list consisting a low risk classification and a high risk classification; presenting the at least one digital scan on a screen of an HMI, according to the classification; and producing, by a report module, in real time, at least one report data element may include diagnostic information, based on the classification of the at least one pathology slide.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic flow diagram, depicting an example for a workflow at a diagnostic pathology institute, according to some embodiments;

FIG. 2 is a block diagram, depicting a computing device which may be included in a system for managing workflow of examination of pathology slides, according to some embodiments;

FIG. 3, is a schematic block diagram, depicting an example of a process of IA-based computational or computer-aided diagnostics (CAD), which may be included in a system for managing workflow of examination of pathology slides, according to some embodiments;

FIG. 4 is a schematic block diagram, depicting a system for managing workflow of examination of pathology slides, according to some embodiments; and

FIG. 5 is a flow diagram, depicting a method of managing workflow of examination of pathology slides, according to some embodiments.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

One skilled in the art will realize the invention may be embodied in other specific forms without departing from the spirit or essential characteristics thereof. The foregoing embodiments are, therefore, to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although some embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although some embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

The term set when used herein can include one or more items. Unless explicitly stated, the method embodiments described herein are not constrained to a particular order or sequence. Additionally, some of the described method embodiments or elements thereof can occur or be performed simultaneously, at the same point in time, or concurrently.

In a first aspect of the invention, some embodiments may include a method and/or a system for managing workflow of examination of pathology slides. As elaborated herein, some embodiments may include one or more modules, implemented in software, hardware or any combination thereof. The one or more modules may be adapted to receive one or more data elements pertaining to a pathology case, originating from one or more data sources, such as outcome of an IA-based pathology examination process, applied to digitally scanned pathology slides. The one or more modules analyze the received one or more data elements and may integrate the one or more data elements as elaborated herein, to provide an integrated workflow of examination of the digitally scanned pathology slides in a pathology institute or laboratory.

As known in the art, computerized decision systems normally include an implicit tradeoff between throughput and accuracy. For example, commercially available decision-support diagnostic tools may provide a hard decision recommendation: a first sample that may have a first score may be labeled as c, whereas a second sample that may have a second score may be labeled as suspicious and may be acted upon (e.g., propagated to a human expert for further analysis). The problem with this approach is that, even though the outcome of such a decision support system (e.g., the score of each sample) may be continuous (e.g., a probability of malignancy, expressed as a continuous percentage), the decision to act upon a sample is always deterministic. Thus, a tradeoff exists between an over-cautious approach that may include excessive marking of samples as suspicious and a precarious approach that may include excessive marking of samples as benign. The first approach may impede the workflow of diagnosticians, whereas the second approach may harm the accuracy of diagnosis.

In a second aspect of the invention, some embodiments of the present invention may produce the integrated workflow such that the workflow may optimize or improve a throughput of a pathology examination process while maintaining or improving a level of accuracy of the pathological examination process.

The term “accuracy” may be used in this context to refer to a level or a percentage at which one or more elements of a diagnosis report of a pathologist may correctly describe a patient's condition as manifested by one or more pathology slides.

The terms “throughput” and “efficiency” may be used in this context interchangeably, to refer to the number of pathology cases and/or slides that one or more pathologists in a pathology institute may examine in a predefined period of time (e.g., as a number of examinations and/or as a percentage of an expected number of examinations).

Some embodiments of the invention may optimize the throughput of a process of examination of pathological slides in view of a predefined required accuracy level, in a sense that they may find a “sweet-spot” in the labeling of one or more elements or regions in one or more slides (e.g., as suspicious) at which the throughput may reach a maximal value and at which the accuracy satisfies the predefined required accuracy level.

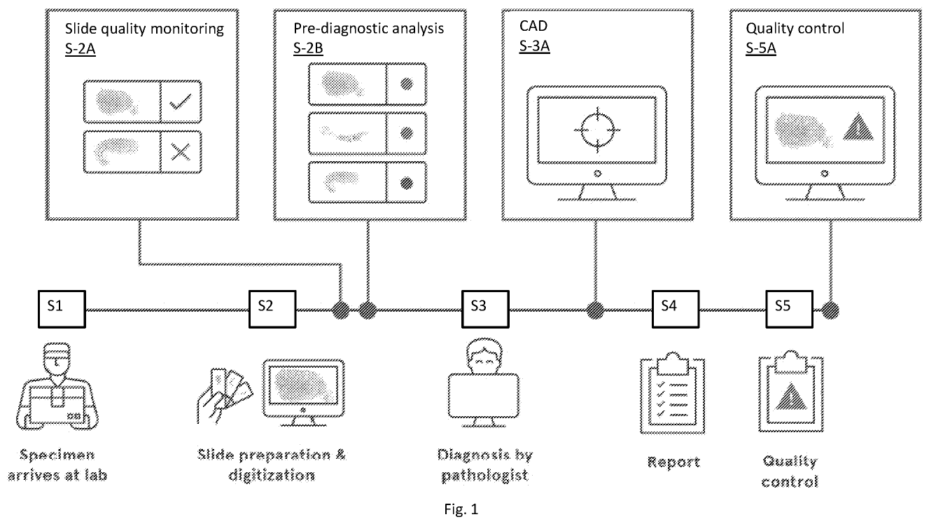

Reference is now made to FIG. 1 which is a flow diagram, depicting an example for a workflow at a diagnostic pathology institute, according to some embodiments. Some elements of the workflow depicted in FIG. 1 may normally be performed in pathology institutes as known in the art. Some embodiments of the present invention may be adapted to improve (e.g., by optimizing) processes depicted in FIG. 1, as elaborated herein.

As shown in step S1, a specimen (e.g., a sample such as a tissue biopsy that may have been ordered by a physician) may arrive at the pathological institute and may be subject to pathological analysis or examination process, as described herein.

In step S2, one or more samples may be processed, to prepare and digitally scan or image (e.g. produce an image of) one or more slides, as normally performed in pathological institutes and as known in the art. The processing of samples may include a plurality of steps, such as: sampling, affixing and cutting of the specimen, to obtain one or more slices that are mountable on one or more glass slides; staining the slides according to requirements of the present clinical pathology case; and performing high-resolution scanning of the slides, e.g. by a WSI scanner, to obtain high resolution images.

As elaborated herein, some embodiments of the invention may optimize a workflow of examination of digitally scanned pathology slides by ensuring that digitally scanned slides are provided to the pathologist according to predefined requirements, so as not to revert to a second process of preparation and examination of the slides.

For example, as shown in step S-2A, some embodiments of the invention may employ digital image processing algorithms to monitor the quality of images (e.g. scanned slides), and mark scanned slides that do not comply with predefined global quality criteria. Such global criteria may include, for example: compliance of the processed sample to requirements of the present clinical pathology case (e.g., performance of specific staining), quality of the glass slide (e.g., amount of dirt, dust, cracks or air bubbles), thickness of the tissue slice, amount of tissue folds and holes in the slice, and features of appearance of the slide (e.g., uniformity of the staining, focus of the scanned image, lighting and/or brightness of the scanned image, contrast of the scanned image, and the like, hereinafter appearance features).

In a second example, as shown in step S-2B and as elaborated herein, some embodiments of the present invention may employ IA-based computational diagnostics algorithms (e.g., a CAD software module) to perform a first reading (e.g., an IA-based, pre-diagnostic examination) of the at least one scanned slide and detect or identify one or more slides that may include visual or morphological features of clinical significance (hereinafter clinical features) and corresponding ROIs therein. Some embodiments of the invention may classify the ROIs and/or slides (e.g., according to scale of severity, as elaborated herein) based on the detected clinical features of the IA-based, pre-diagnostic examination.

Some embodiments of the invention may subsequently produce a workflow, according to which a pathology slide may be prepared for a pathologist examination based on the classification of ROIs and/or slides. Additionally, or alternatively, the produced workflow may include an order of presentation or a guidance of view to the pathologist through ROIs of the specific pathology case (e.g., based on classification of ROIs and/or slides to classifications of suspected severity).

As shown in step S3, some embodiments of the invention may present the slides on a user interface (UI) or a human-machine interface module, such as a Digital Pathology Workflow (DPW) module, to a selected pathologist or pathology expert, who may examine the one or more scanned slides to detect clinical features therein.

As shown in step S-3A, some embodiments of the invention may be configured to perform a concurrent examination of the scans of the present pathological case by an IA-based computational diagnostics algorithms (e.g., a CAD software module), parallel to the examination performed by the pathological expert. Some embodiments of the invention may thus integrate between a first examination process, performed by a human expert and a second examination process, performed by an IA-based algorithm, as elaborated herein.

As shown in step S4, the pathologist may produce an examination report that may include an outcome of his or her examination of the at least one scanned slide. Some embodiments of the invention may integrate the process of producing the examination report with the first (human expert) examination process and second (IA-based) examination process, to associate between elements or findings of the first and/or second examination processes and elements of the produced examination report, as elaborated herein.

As shown in step S5, some embodiments of the present invention may be adapted to detect one or more discrepancies between different data elements pertaining to a pathology case and/or slide, and may (as shown in step S5-A) produce at least one notification for detection of such a discrepancy.

According to some embodiments of the invention, the invention may apply rule-based logic to determine a validity of the examination report. For example, in a condition that data pertaining to a pathology case (e.g., originating from an LIS database), such as a previous physician's report or a previous test result (e.g., including medical findings) does not comply with the produced report (e.g., devoid of the medical findings), some embodiments of the invention may produce a notification of this discrepancy, indicating, for example, that a currently examined tissue fragment may have been obtained through an erroneously-located biopsy procedure.

Additionally, or alternatively, some embodiments of the present invention may integrate between a first examination process, performed by a human expert, a second examination process, performed by an IA-based algorithm and a process of producing the examination report, in order to determine a validity of the process of examination by the human expert, and/or validity of the examination report. For example, some embodiments of the invention may detect a condition in which the pathology examination procedure was erroneous or incomplete. Some embodiments may subsequently (as shown in step S5-A) produce at least one notification for such error.

For example, some embodiments of the invention may track presentation properties (e.g., pan, zoom, contrast, etc.) that an examiner may select during their examination of a digitally scanned pathology slide. In a condition that a pathology expert has reported (in the examination report) that no clinical findings were detected in a pathology slide while the IA-based examination has produced suspicious clinical findings, some embodiments of the invention may determine whether: (a) the human expert has viewed the relevant ROIs and decided they are of no consequence (and thus the examination report may be valid); or (b) the human expert has missed the relevant ROIs (and thus the examination report may be invalid).

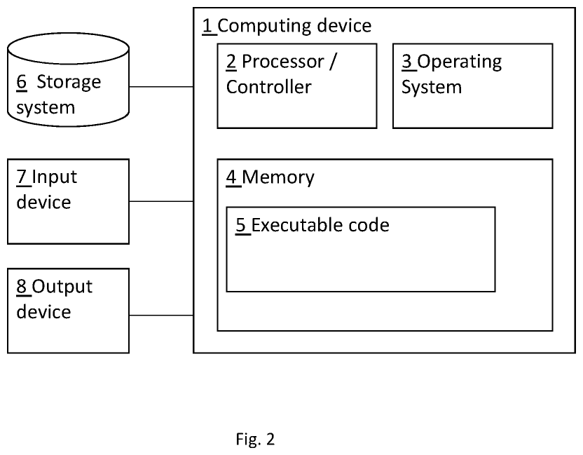

Reference is now made to FIG. 2, which is a block diagram depicting a computing device which may be included in a system for managing workflow of examination of pathology slides, according to some embodiments.

Computing device 1 may include a controller 2 that may be, for example, a central processing unit (CPU) processor, a graphics processing unit (GPU) processor, a chip or any suitable computing or computational device, an operating system 3, a memory 4, executable code 5, a storage system 6, input devices 7 and output devices 8. Controller 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to some embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of Computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of, possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by controller 2 possibly under control of operating system 3. For example, executable code 5 may be an application that may manage workflow of examination of pathology slides as further described herein. Although, for the sake of clarity, a single item of executable code 5 is shown in FIG. 1, a system according to some embodiments of the invention may include a plurality of executable code segments similar to executable code 5 that may be loaded into memory 4 and cause controller 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to examination of pathology slides may be stored in storage system 6 and may be loaded from storage system 6 into memory 4 where it may be processed by controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of CPUs or any other suitable multi-purpose or specific processors or controllers (e.g., controllers similar to controller 2), a plurality of input units, a plurality of output units, a plurality of memory units, and a plurality of storage units.

The term “Neural Network” (NN) or artificial neural network (ANN), such as a NN adapted to implement an algorithm of machine learning, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. One or more processing units (e.g., one or more CPUs, GPUs, or a dedicated hardware devices) may perform the relevant calculations.

Some embodiments of the invention may include one or more ML-based modules that may be or may include specific implementations of NNs, as known in the art. For example, some embodiments of the invention may include one or more ML modules that may be or may include Convolutional Neural Networks (CNNs), adapted to implement functions of image analysis, including for example, classification of one or more regions of digitally scanned slides according to classifications of severity, as elaborated herein.

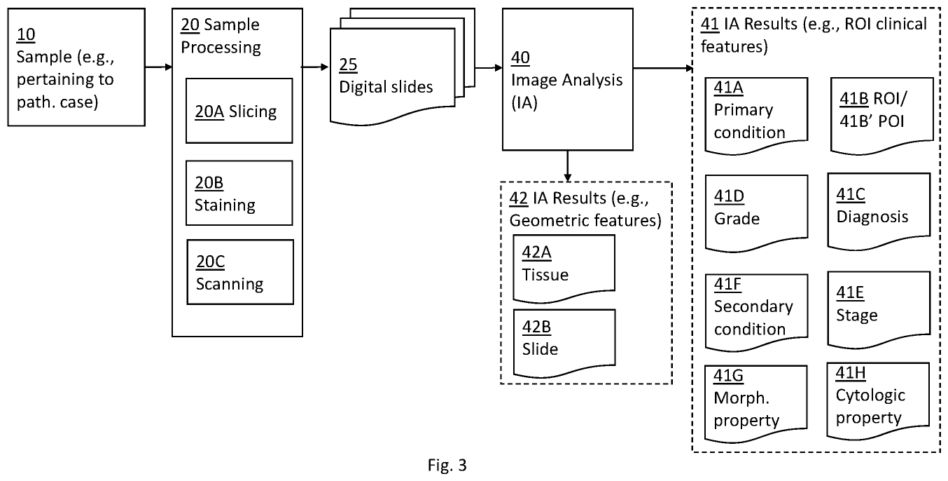

Reference is now made to FIG. 3, which is a is a schematic block diagram, depicting an example of a process of IA-based computational diagnostics or CAD, which may be included in a system for managing workflow of examination of pathology slides, according to some embodiments.

As elaborated herein, in relation to FIG. 1, a workflow of a pathological institute may include receiving a specimen or a sample 10 of a tissue, associated with a specific pathological case. The tissue may be processed by one or more processing modules 20 (e.g., 20A, 20B, 20C), according to predefined requirements, associated with the type of the tissue and a suspected clinical condition, to obtain one or more digitally scanned slide images 25.

For example, sample 10 may be: sliced to a predefined thickness by a slicing module 20A; stained according to predefined clinical requirements or protocols by a staining module 20B, where the predefined clinical requirements may be dictated according to specifications of the clinical pathology case (e.g., a suspected type of cancer); and digitally scanned according to predefined configurations (e.g., resolution, brightness, contrast, etc.) by a high-resolution scanning module (e.g., a WSI scanner) 20C.

Some embodiments of the invention may include a IA computational diagnostics module (hereinafter IA module) 40. IA module 40 may be adapted to receive one or more digitally scanned slides 25 from the one or more processing modules 20 (e.g., 20A, 20B and 20C). IA module 40 may employ one or more algorithms to extract one or more clinical features, and produce one or more diagnostic results 41 (e.g., 41A, through 41H). The one or more diagnostic result 41 of IA module 40 may consequently be utilized by an embodiment of a system for managing workflow of examination of pathology slides, as elaborated herein.

According to some embodiments, IA module 40 may be implemented as a computational module, that may be executed by a processor (e.g., element 2 of FIG. 2), to produce the one or more diagnostic results 41. For example, IA module 40 may be or may include an artificial-intelligence based application, adapted to train a NN such as a deep NN, a convolutional NN and/or any other computational module known in the art, to extract at least one clinical feature of the scanned slide.

For example, IA module 40 may be configured to extract or detect at least one primary clinical feature, that may indicate a high priority medical condition 41A (e.g., a medical condition of primary importance or priority such as existence of a malignant tumor, indication of a perineural invasion, etc.). The at least one high priority condition 41A may include, for example: a morphological or cellular feature (e.g., a shape of one or more cells, a size of one or more cells, and the like), a pattern of a stain or dye absorbed by the examined tissue, and the like.

In another example, IA module 40 may be configured to extract or detect at least one ROI 41B, that may be suspected to include at least one extracted high priority condition 41A, and should, therefore, be studied carefully by an expert pathologist.

In another example, IA module 40 may be configured extract or detect at least one secondary clinical feature or medical condition 41F, such as a clinical condition of secondary importance or priority such as a pre-cancerous condition, an inflammation, etc.

In another example, IA module 40 may be configured to extract or detect at least one ROI 41B, that may be suspected to include at least one extracted secondary clinical feature or medical condition 41F.

In another example, IA module 40 may be configured to extract or detect at least one suggested diagnosis 41C (e.g., a malignant tumor of a particular type), corresponding to an extracted high priority condition 41A and/or respective detected ROI 41B.

In another example, IA module 40 may be configured to extract or detect at least one suggested diagnosis 41C (e.g., an inflammation), corresponding to a detected secondary medical condition 41F and/or respective detected ROI 41B.

In another example, IA module 40 may be configured to extract or detect at least one grade 41D and/or stage 41E of a cancerous tumor, as per the suggested diagnosis 41C and/or respective detected ROI 41B, as known in the art of oncological medicine.

In another example, IA module 40 may be configured to extract or detect at least one suggested secondary finding 41, corresponding to the extracted secondary clinical feature or medical condition 41F (e.g., an inflammation, a pre-cancerous condition, and the like).

In another example, IA module 40 may be configured to extract or detect at least one morphologic property 41G corresponding to a high priority medical condition 41A, a secondary medical condition 41F, and/or a respective ROI 41B.

In yet another example, IA module 40 may be configured to extract or detect at least one cytologic property 41H corresponding to a high priority medical condition 41A, a secondary medical condition 41F, and/or a respective ROI 41B.

An embodiment of the present invention may utilize one or more results 41 (e.g., 41A through 41H) to optimize the process of diagnosis by a pathology expert (e.g. by controlling and modifying sample pre-processing 20, prioritizing scanned slides according to primary and secondary findings and performing triage of pathological cases), as explained herein.

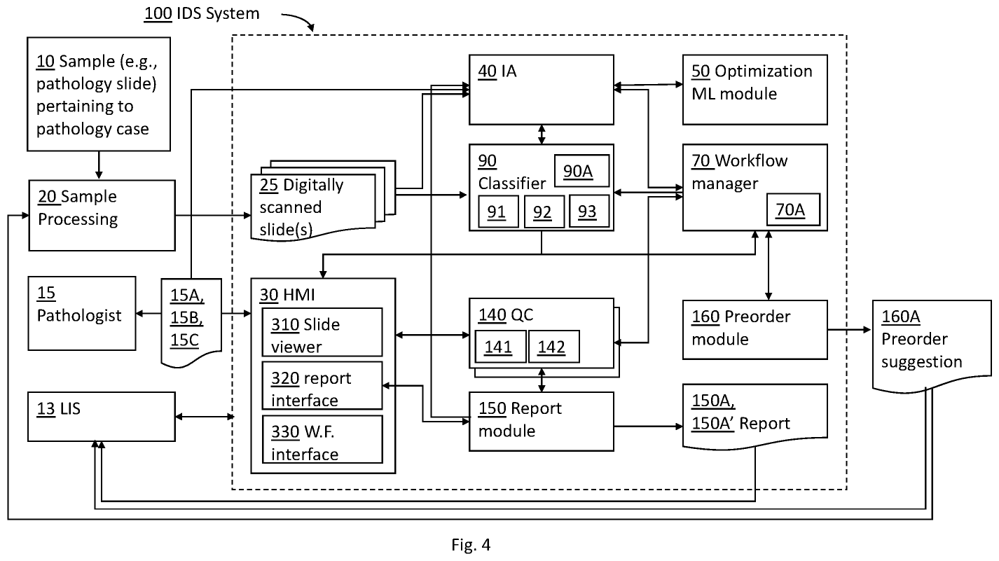

Reference is now made to FIG. 4, which is a schematic block diagram, depicting an Integrated Diagnosis System (IDS) 100 for managing workflow of examination of pathology slides, according to some embodiments. According to some embodiments of the invention, IDS 100 may be implemented as a software module, a hardware module or any combination thereof. For example, IDS 100 may be or may include a computing device (such as element 1 of FIG. 2), and may be adapted to execute one or more modules of executable code (e.g., element 5 of FIG. 1) to managing workflow of examination of pathology slides, as elaborated herein. In another example, one or more modules of IDS 100 (e.g., modules 20, 30, 40, 50, 70, 90, 140, 150, 160) may be implemented or executed by one or more computing devices (such as element 1 of FIG. 2).

According to some embodiments of the invention, IDS 100 may receive a sample 10, such as a pathology slide. IDS 100 may include a sample processing module 20, configured to perform at least one operation of slice preparation such as slide slicing 20A staining 20B and/or scanning 20C, as elaborated herein (e.g., in relation to FIG. 3). Additionally, or alternatively, and as depicted in the example of FIG. 4, IDS 100 may be communicatively connected (e.g., via a computer communication network such as the internet) to an external sample processing module 20, which may not be included in IDS 100.

According to some embodiments of the invention, IDS 100 may include an IA module 40, configured to receive at least one digital scan 25 of at least one pathology slide 10 related to a clinical pathology case. IA module 40 may perform at least one algorithm of image analysis on the received digitally scanned slide 25 to extract at least one clinical feature of the at least one slide.

For example, the at least one extracted clinical feature may be an IA output element 41 as elaborated herein (e.g., in relation to FIG. 3). For example, the at least one extracted clinical feature may include one or more of: a primary clinical feature or high priority medical condition 41A, a secondary medical condition or finding 41F, one or more ROIs 41B corresponding to the high priority medical condition, one or more ROIs 41B corresponding to the secondary medical condition or findings 41F, a diagnosis 41C of the high priority medical condition 41A, a diagnosis 41C of the secondary medical condition or finding 41F, a grade 41D of the high priority medical condition 41A, a stage 41E of the high priority medical condition 41A, a morphologic property 41G of the high priority medical condition 41A, and a cytologic property 41H of the high priority medical condition 41A.

Additionally, or alternatively, IDS 100 may be communicatively connected (e.g., via a computer communication network such as the internet) to an external (e.g., a third-party) IA module 40, which may not be included in IDS 100, and may receive the one or more IA outputs 41 (e.g., 41A through 41F) this communication.

According to some embodiments of the invention, IA module 40 may receive continuous (e.g., repetitive, with each examined pathology case) data from a human expert pathologist 15, such as expert data element 15B, pertaining to diagnosis of the presented pathology slide 25. Additionally, or alternatively, IA module 40 may receive continuous (e.g., repetitive, with each examined pathology case) data pertaining to a pathology report 150A (e.g., from a report module 150, as elaborated herein). IA module 40 may use the received data (e.g., from the pathologist 15 and/or from the report 150A) as supervisory data, to further train and improve the extraction of clinical features 41 and/or prediction or detection of IA 40 results.

According to some embodiments, IDS 100 may include a classifier module 90, adapted to receive one or more IA output element 41 (e.g., a diagnosis 41C, a grade 41D a stage 41E and a corresponding ROI 41B) pertaining to a specific digitally scanned pathology slide 25. Classifier module may be adapted to classify or associate specific slide 25 to a classification of a set of severity classifications (e.g., malignant, borderline and benign), based on at least one output data element of IA 40 such as clinical features 41 (e.g., elements 41A through 41F of FIG. 3).

For example, the set of classifications may include a low risk classification (e.g., a classification corresponding to benign findings of pathological cases and/or slides), a borderline classification (e.g., a classification corresponding to pre-cancerous cells, non-cancerous clinical findings that may require treatment, or borderline results indicating potentially, though inconclusive, suspicious findings) and a high risk classification (e.g., a classification corresponding to cancerous or malignant clinical findings).

According to some embodiments, classifier module 90 may be an ML-based module, trained to receive one or more input data elements from IA 40 corresponding to a specific scanned slide 25 and classify the slide to the one or more severity classifications. According to some embodiments, classifier module 90 may receive one or more input data elements from a human pathology expert (e.g., via HMI 30, as elaborated herein) and may be trained through a supervised training process, having the received one or more input data elements as training feedback.

According to some embodiments, classifier module 90 may classify or associate different elements pertaining to the clinical pathology case with the one or more severity classifications.

For example, classifier module 90 may classify one or more ROIs 41B of a slide 25 with a specific severity class. This type of classification may herein be referred to as ROI classification 91.

Additionally, or alternatively, classifier module 90 may associate a slide 25 with a specific severity classification, based on classification of ROIs included in the slide. For example, a slide may be associated with the most severe ROI classification 91 included therein. This type of classification may herein be referred to as slide classification 92.

Additionally, or alternatively, classifier module 90 may associate a clinical pathology case with a specific severity classification, based on classification of slides 25 pertaining to the case. For example, a clinical pathology case may be associated with the most severe slide classification 92 included therein. This type of classification may herein be referred to as case classification 93.

As shown in the example of FIG. 4, IDS 100 may include an HMI 30 such as a DPW module, adapted to present the at least one digitally scanned slide 25 on a screen to a selected pathologist or pathology expert 15, who may examine the at least one digitally scanned slide 25 to detect clinical features therein.

According to some embodiments, HMI 30 may include a slide viewer module 310, configured to receive (e.g., from a user), at least one viewing context data element. The viewing context data element may include, for example, a selection or identification (e.g., a number) of a pathology case, a selection or identification (e.g., a number) of a specific digitally scanned pathology slide 25, a selection of one or more ROIs and one or more viewing properties such as a selection of panning, a selection of zoom, a selection of brightness and a selection of contrast of a required presentation. Slide viewer module 310 may present the digitally scanned pathology slide 25 on the screen, according to the at least one viewing context data element.

For example, HMI 30 may enable a user (e.g., a pathologist) to select an identification (e.g., a number) or a representation (e.g., an icon or a minimized image) of an ROI 41B. Slide viewer 310 may respond to the user's selection by presenting the respective ROI 41B on the middle of the screen, at an appropriate zoom level, and optionally highlight or encircle the relevant ROI 41B (e.g., in a color that may represent the ROI's 41B classification).

According to some embodiments, HMI 30 may include a report interface 320, adapted to communicate with report module 150. Report interface 320 may present (e.g., on the screen of HMI 30), adjacent to selected ROI 41B, one or more data elements of a pathology report pertaining to selected ROI 41B, as elaborated herein.

In another example, HMI 30 may enable a user (e.g., a pathologist) to select at least one pathology slide 25. Slide viewer 310 may respond to the user's selection by presenting the respective pathology slide, and may include in the presentation one or more marked, highlighted or encircled ROIs 41B included in slide 25. Additionally, report interface 320 may present (e.g., on the screen of HMI 30), one or more data elements of a pathology report pertaining to selected slide 25, as elaborated herein.

In another example, HMI 30 may enable a user (e.g., a pathologist) to select a pathology case. Slide viewer 310 may respond to the user's selection by presenting one or more representations (e.g., icons or minimized images) of slides 25 pertaining to the case. Additionally, report interface 320 may present (e.g., on the screen of HMI 30) one or more data elements of a pathology report pertaining to the case and or one or more LIS data elements 13 pertaining to the case.

As elaborated herein, some embodiments of the invention may integrate functionality of HMI 30 with that of classifier module 90 in a plurality of aspects, such that the one or more digitally scanned pathology slides 25 may be presented to, and examined by a human examiner on HMI 30 based on the output of classification module 90.

As a basic example, the one or more slides 25 and or one or more ROIs 41B may be ordered for presentation and/or examination on HMI 30 according to their respective severity (e.g., malignant severity ROIs 41B and/or slides 25 before benign severity ROIs 41B and/or slides 25).

Additional aspects of integration between classifier module 90 and HMI 30 are elaborated herein.

As elaborated herein (e.g. in relation to FIG. 1), an outcome of a pathological examination typically includes a report issued by an expert pathologist 15.

According to some embodiments, and as shown in the example of FIG. 4, IDS 100 may include a report module 150, adapted to produce, in real time or in near-real time, at least one report data element 150A that includes diagnostic information, based on the classification of the at least one pathology slide. For example, the at least one report data element 150A may include a location of a specific ROI 41B within a slide 25, and a respective suspected diagnosis 41C, grade 41D and/or stage 41E associated with the specific ROI 41B.

As elaborated herein, some embodiments of the invention may integrate functionality of report module 150 with functionality of HMI 30 and of classifier module 90 in a plurality of aspects, to improve efficiency or throughput, as well as accuracy of a workflow of pathology examination in relation to currently available systems of digital pathology.

As a basic example, slide viewer module 310 may present the digitally scanned pathology slide 25 on the screen, in conjunction with at least one diagnostic report data element 150A and/or at least one ROI classification 91 data element, according to the at least one viewing context data element 15A. For example, if viewing context data elements 15A such as zoom and/or a panning are selected by a pathologist 15 so as to enable viewing of a specific ROI 41B on the screen, then slide viewer module 310 may present the relevant portion of the slide according to the viewing context data element 15A, alongside relevant indications (e.g., colorization) of ROI, slide and/or case classification 93, and/or relevant diagnostic report data elements 150A (e.g., report data elements 150A pertaining to ROIs 41B in the context of the currently viewed portion of slide 25). Such context-based, concurrent presentation of (a) severity classification of classifier module 90, (b) relevant portions of a report 150A and (c) segments of the scanned slide 25, may enable a pathologist 15 to concurrently view all relevant elements of the slide required for the examination process and may facilitate an interactive, swift approach for producing report 150A.

In another example, report module 150 may produce report 150A iteratively, by combining input from a pathologist 15 (e.g., via HMI 30) and from classification module 90. For example, in a first iteration, classifier module 90 may indicate a specific ROI 41B as having borderline severity classification. Report module 150 may produce a portion of report 150A including clinical data (e.g., elements 41A through 41F) pertaining to the relevant ROI 41B, and a slide viewer module 310 of HMI 30 may present the slide 25 on a screen, and highlight or direct a pathologist 15 to examine the relevant ROI 41B.

In a second iteration, HMI 30 may receive, from a user, at least one expert data element 15B pertaining to diagnosis of the presented pathology slide 25. For example, a report interface module 320 of HMI 30 may prompt the pathologist 15 to indicate or provide his or her professional opinion 15B regarding the relevant ROI 41B. The examiner may, for example, indicate 15B that the ROI 41B is in fact benign or malignant. Subsequently, report module 150 may modify report 150A, so as to integrate the at least one expert data element 15B into the at least one report data element 150A and produce an integrated report 150A′ data element.

The term “integrated” may be used in this context to indicate an integration or combination of automatically received data originating from classifier module 90 (e.g., a severity class) and/or IA 40 (e.g., clinical features 41) with professional opinion data 15B obtained from the pathology examiner, which may reflect the pathologist's 15 decision. It may be appreciated that report data elements 150A and 150A′ may or may not be identical, depending on data 15B received from the pathologist 15.

According to some embodiments, HMI 30 may be configured to present the integrated report data element 150A′ on a screen according to the context of the currently presented portion of slide 25 (e.g., according to the at least one received viewing context data element 15A), in a similar manner as discussed herein in relation to report data element 150A.

According to some embodiments, classifier module 90 may receive professional opinion data element 15B from the pathologist 15 (e.g., inline, during the process of examination) as training feedback, and may use data element 15B to further improve classification of severity.

It may be appreciated that the workflow described in the above example may include a plurality of improvements over currently available systems of digital pathology.

For example, one or more ROIs 41B may be associated with at least one respective extracted clinical feature 41 (e.g., elements 41A through 41F) and/or with at least one respective ROI and/or slide classification 92. HMI 30 may be configured to produce a viewing suggestion 15C according to at least one of: the one or more ROIs 41B, the at least one clinical feature 41 and the slide and/or ROI classification 91. In some embodiments, viewing suggestion 15C may be presented to a user (e.g., a pathologist 15) for use at his or her discretion. Additionally, or alternatively, slide viewer module 310 may be adapted to present slides 25 and/or ROIs 41B according to viewing suggestion 15C. For example, slide viewer module 310 may be adapted to present slides 25 and/or ROIs 41B according their respective severity classification, and indicate (e.g., highlight by color) important areas within a slide 25, so as not to waste time on lesser clinical features 41, ROIs and/or slides of the case.

In another example, slide viewer module 310 may keep track of the actions of a pathologist (e.g., panning, zooming, etc.) so as to ensure that the pathologist 15 does not miss important regions of interest 41B within slides of the pathology case.

In another example, as elaborated herein, report module 150 may automatically prepare one or more portions of a report 150A in one or more iterations, and include relevant input from IA 40 and/or classifier module 90 as a ‘scaffold’ for a report. Report module 150 may integrate the data from IA 40 and/or classifier module 90 with one or more diagnostic opinion data elements 15B from the pathologist 15 (e.g., via report interface 320), so as to produce an integrated report 150A′ in a manner that is time-wise efficient.

Moreover, it may be appreciated that report interface 320 may integrate functionality of report module 150, slide viewer 310, IA 40 and/or classifier module 90, to detect and/or prevent human errors. For example, in currently available systems for examination of pathology slides, human pathologists may perform one or more errors that may not necessarily be related to misdiagnosis. This may include for example examining a first slide but reporting the findings as related to a second slide. As explained herein, some embodiments of the present invention (e.g., report interface 320) may produce a report (or a report scaffold) automatically, in the context of, or in conjunction with the examination of a specific slide or a specific portion of a slide. Therefore, some embodiments of the invention may provide an improvement over currently available systems by detecting and/or bypassing such human errors.

In another example, some embodiments of the invention may enhance uniformity and/or consistency of reports 150A, among different pathologists and among different reports produced by the same pathologist. It may be appreciated that currently available systems for examination of pathology slides may not guide specific pathologists through the fields and data elements that should be included within the pathology report. Subsequently, current practice may result in non-standard reports, that may lack important information.

In contrast, some embodiments of the invention may include a workflow interface module 330 adapted to interface or collaborate with workflow manager 70, as elaborated herein. According to some embodiments, workflow interface module 330 may be configured to guide pathologists through the process of producing the report 150A. Such guidance may, for example, produce standardized, comparable reports 150A that may include all required information (e.g., fields of data elements). Additionally, said guidance may produce reports 150A that may clearly associate findings (e.g., pathological findings) to specific circumstances (e.g., location of specific ROIs, data elements pertaining to the examination process, data elements pertaining to the patient and/or data elements pertaining to the examiner).

Additionally, or alternatively, and in contrast to currently available systems for examination of pathology slides, some embodiments of the invention (e.g., report interface 320) may automatically produce report 150A (or a scaffold of a report 150A) that may include all predefined data elements or fields (e.g., tumor size measurements, quantification of mitoses, etc.), and may thus avoid producing reports with lacking data and/or with findings that are reported inconsistently (e.g., using different terms, or different types of measurements, such as tumor size vs. tumor percent).

In another example, input from the pathologist 15 via report interface 320 may be utilized inline to further train classifier module 90.

In another example, report module 150 may include, within report 150A, an association of specific examined ROIs 41B with respective pathologist 15 opinion for the purpose of quality control. For example, in a condition that a pathologist 15 has erroneously determined a case as benign, a retrospective, second examination of ROIs 41B of the case may reveal the cause for error. For example, the second examination may determine whether the pathologist 15 has failed (e.g., negligently) to examine all ROIs 41B of a case (e.g., at the required magnification level and/or for a reasonable time duration), or whether a benign-diagnosed ROI 41B turned out eventually to be malignant, in which case both the pathologist 15 and classifier module 90 may be further trained according to the finding of the retrospective examination.

As known in the art, pathologists are often required to assess a quantity or measurement of a tissue-related property (e.g., a geometric property) of a tissue in a scanned pathology slide 25.

As elaborated herein, some embodiments of the invention may provide an improvement over currently available digital pathology systems by: (a) performing the required measurements or assessments automatically (e.g., without requiring involvement of the pathologist 15), (b) performing the required measurements or quantification accurately (e.g., by means of computerized algorithms, rather than by manual measurement or assessment, as currently performed in pathology institutions), and (c) performing the required measurements or quantification in a manner that is integrated within the workflow, as elaborated herein.

According to some embodiments, IA module 40 may be configured to automatically extract (e.g., by applying one or more algorithms of image processing) at least one tissue-related property 42A pertaining to one or more tissue fragments of at least one pathology slide 25. The at least one property 42A may include one or more properties of measurement and/or quantization that a pathologist may need to provide in order to complete their pathology report 150A′.

For example, the at least one tissue-related 42A property may include: a geometric property such as a length of a tissue fragment, a shape of a tissue fragment, an area of a tissue fragment, a volume of a tissue fragment and the like. Additionally, or alternatively, the at least one tissue-related 42A property may include one or more quantitative tissue-related 42A properties, such as a number of tissue fragments in the scanned pathology slide 25.

Some embodiments may integrate the automatic extraction of tissue-related properties 42A with the preparation of integrated report 150A′. For example, report module 150 may receive one or more automatically extracted tissue-related property 42A, and include that property (e.g., a number of tissue fragments in a slide 25 and/or in a plurality of slides pertaining to a case) in the produced report 150A′ (e.g., indicate each section in the report as pertaining to a specific tissue fragment of the overall number of tissue fragments).

Additionally, or alternatively, some embodiments of the invention may utilize the automatic extraction of tissue-related properties 42A to facilitate an automated process of quality control (QC).

For example, IDS 100 may include a QC module 140, adapted to receive one or more metadata elements from sample processing module 20 (which, as elaborated herein, may or may not be included in IDS 100). According to some embodiments, the one or more metadata elements may include descriptive information regarding the processed samples. The descriptive information of sample processing module 20 may include, for example, one or more tissue-related properties relating to the samples 10 as they were processed in the lab, and/or as indicated (e.g., in a text file) by a physician who has extracted sample or biopsy 10. Such descriptive information may include, for example, a number of tissue fragments, a length of a tissue fragments, a shape of tissue fragments, a staining of a slide (e.g., of tissue fragments included therein) etc.

According to some embodiments, QC module 140 may analyze at least one tissue-related property of a tissue fragment pertaining to a specific pathology case, in order to determine a discrepancy between metadata of one or more scanned pathology slides of the specific pathology case, and the tissue-related property. For example, QC module 140 may compare the descriptive information of sample processing module 20 with the one or more extracted tissue-related properties 42A, to ensure that all tissue fragments pertaining to the clinical pathology case have been received for examination by a pathologist 15.

Additionally, or alternatively, QC module 140 may receive from slide viewer 310 an account of the pathologist 15 viewing actions (e.g., viewing context data elements 15A selected by the pathologist 15), corresponding to the portions of slides that have been studied by the pathologist 15. QC module 140 may compare the account of the pathologist 15 viewing actions from slide viewer 310 with the one or more extracted tissue-related properties 42A, to ensure that the pathologist 15 has indeed studied all tissue fragments pertaining to the pathology case.

For example, in a condition that AI 40 extracts a clinical feature 41 that may be or may include a high priority, or primary medical condition 41A (e.g., malignancy), associated with a corresponding ROI 41B (e.g., a region of a tumor), and the account of the pathologist 15 viewing actions does not show that pathologist 15 has viewed the relevant area using a predefined adequate magnification or zoom, QC module may produce a corresponding notification, and HMI 30 may be configured to present the notification via slide viewer 310.

According to some embodiments, IA module 40 may be configured to automatically extract (e.g., by applying one or more algorithms of image processing) at least one slide-related property 42B, associated with the at least one extracted clinical feature 41, and pertaining to a pathology slide 25.

The at least one slide-related property 42B may include, for example: a location of an extracted clinical feature 41 within the slide, and/or a quantification of the extracted clinical feature within the slide.

For example, in a condition that the extracted clinical feature 41 is a morphologic property 41G or a cytologic property 41H of a cell included in scanned slide 25, IA 40 may be configured to extract a quantity and/or a location of cells having similar morphologic properties 41G or cytologic properties 41H within the relevant one or more digitally scanned slides 25.

In another example, IA 42 may extract (e.g., using an image processing algorithm) a slide-related property 42B which may correspond to a dimension (e.g., a length, a volume, etc.) of a tumor (e.g., across one or more digitally scanned pathology slides).

In another example, IA 42 may extract (e.g., using an image processing algorithm) a slide-related property 42B which may correspond to a tumor percentage (e.g., a number or percentage of cancerous cells or cancerous ROIs 41B within an overall tissue area on one or more digitally scanned pathology slides) and/or a percentage per Gleason score.

In another example, IA 42 may extract an indication of a cancer grade 41D which, as known to persons skilled in the art, typically relies on cytologic and/or morphologic features, and may also include one or more cell quantification data elements.

In another example, IA 42 may extract (e.g., using an image processing algorithm) a slide-related property 42B which may correspond to a mitosis count (which, as known in the art, may indicate cell proliferation and hence proliferation of cancer within a tissue).

In another example, IA 42 may extract (e.g., using an image processing algorithm) a slide-related property 42B which may correspond to an indication of a distance between cancerous cells and a predefined margin or border of a tissue and/or an indication of whether cancerous cells have metastasized beyond the predefined border (which, as known in the art, may also correlate to a stage 41E of a tumor).

In yet another example, IA 42 may extract (e.g., using an image processing algorithm) a slide-related property 42B which may correspond to an IHC quantification which, as known in the art, may indicate or assess the expression of cellular proteins in a cancerous tissue, or assist in the diagnosis and/or grading of various conditions.

Some embodiments of the invention may integrate the automatic extraction of slide-related properties 42B with the preparation of integrated report 150A′. For example, report module 150 may receive one or more data elements of slide-related properties 42B (e.g., data pertaining to quantity and/or location of extracted clinical features 41 and corresponding ROIs 41B) and may include these one or more data elements in the produced report 150A′.

Additionally, or alternatively, some embodiments of the invention may integrate the automatic extraction of slide-related properties 42B with functionality of HMI 30. For example, slide viewer 310 of HMI 30 may receive one or more data elements pertaining to quantity and/or location of corresponding extracted clinical features 41, and may present a portion of a digitally scanned slide 25 on a screen, where the clinical features 41 corresponding to the automatically extracted slide-related properties 42B may be highlighted (e.g., colored) so as to clearly present relevant instances of clinical feature 41 to the pathologist 15.

According to some embodiments, IA module 40 may be configured to automatically extract (e.g., by applying one or more algorithms of image processing) a value of at least one slide-related property 42B that corresponds to a quality of a digitally scanned slide 25.

For example, IA module 40 may identify a condition in which a digitally scanned slide 25 is improperly focused, improperly stained, contains an unusual amount of tissue (e.g., insufficient or abundant), contains poorly cut tissue fragments, and the like. Accordingly, slide-related data elements 42B may include an indication of said properties of quality of the digitally scanned slide 25 (e.g., an indication of impaired focus, an indication of improper staining, etc.). Some embodiments of the invention may subsequently utilize slide-related data elements 42B to preorder at least one operation (e.g., at least one additional slide 25) from sample processing unit, as elaborated herein (e.g., in relation to preorder module 160) so as to have the information ready for the pathologists and save turn-around time.

According to some embodiments of the invention, IDS 100 may be communicatively connected with or may include an LIS module 13. LIS module 13 may be or may include a computing device (e.g., element 1 of FIG. 2) associated with or including a database (e.g., element 6 of FIG. 2).

According to some embodiments, LIS module 13 may maintain or store clinical data, metadata and/or workflow information associated with one or more pathological cases, including for example: clinical parameters associated with a patient (e.g., lab test results, gender, age, weight, prescriptions, chronic diseases, family history, etc.); notes of a treating physician (e.g., notes that pertain to a suspected diagnosis); a report of a radiologist; a historical biopsy report associated with the patient; a type of tissue associated with the slide.

Additionally, or alternatively, LIS 13 may include data pertaining to each prepared slide 25, including for example, the association and ordering of each slide within a pathology sample or case 10, a stain type of each slide, associating between IHC slides and H&E slides.

Additionally, or alternatively, LIS 13 may include data pertaining to the pathology institute and/or one or more pathologists 15 therein. For example, the pathology institute related information may include: a field of specialization of a pathologist (e.g. a specific pathologist of a plurality of pathologists); a number of pathological cases assigned to each pathologist; history of circumstances of pathological examinations (e.g. time of day, general workload, time spent on each examination, and the like) per each pathologist; and an availability of each pathologist of the plurality of pathologists.

According to some embodiments, HMI 30 may be configured to present (e.g., on a screen, via slide viewer 310) one or more LIS 13 data elements (e.g., as elaborated above), in conjunction with, and/or in the context of a presented, relevant slide 25.

According to some embodiments of the invention, IDS 100 may include one or more QC modules 140 adapted to detect and/or indicate detection of a discrepancy between information included or stored by LIS 13 and one or more elements of report 150A′.

For example, QC modules 140 may be configured to receive at least one LIS 13 data element pertaining to the clinical pathology case and to analyze the integrated report data element 150A′ in view of the at least one LIS data element to detect at least one discrepancy. QC modules 140 may subsequently produce a notification data element for HMI 30, which may, in turn, by configured to present the relevant notification of the detected at least one discrepancy on a screen of slide viewer 310.

For example, in a condition that the LIS information includes a textual data element describing a physician's report of a mammography scan, and the report includes indications of suspected lesions and/or areas of calcification, QC 140 may be adapted to extract words of interest (e.g., mammogram, calcification, lesion, BRCA (Breast Cancer gene), etc.) from the physician's report. For example, in a condition that the information included within the physician's report is stored structurally, QC 140 may extract the required words according to the predefined structure. Additionally, or alternatively, QC 140 may include or may employ at least one sub-module, such as a natural language processing (NLP) module 141, adapted to extract the words of interest.

QC 140 may then analyze integrated report 150A′ in order to ascertain whether corresponding information is found there (e.g., codes for breast cancer diagnosis, morphology and/or grade). If no relevant information is found in integrated report 150A′, QC 140 may produce a notification data element 142, indicating that the biopsy or sample 10 of the current pathological case may have missed the suspected breast region, and HMI 30 may present the produced notification data element 142 via slide viewer 310 as a warning to the pathologist 15.

In another example, QC 140 may identify a discrepancy between one or more first data elements identified by IA 40 on the slide and one or more second data elements received from LIS 13. As a simple, non-limiting example, an examined slide 25 may be indicated (e.g., in LIS 13) as originating from a breast biopsy, whereas IA 40 (e.g., tissue data element 42A) may identify the tissue type as originating from a gastric tract. It may be appreciated that QC 140 may be adapted to identify many other types of such discrepancies, pertaining to specific types of tissues and pathology cases.

According to some embodiments of the invention, IDS 100 may include a workflow manager module 70, adapted to control a workflow of diagnosis of one or more a pathology cases, such as elaborated herein, e.g., in relation to FIG. 1. For example, as elaborated herein, workflow manager module 70 may enable or allow a pathologist to access data elements pertaining to samples and/or cases 10, slides 25, ROIs 41B and relevant data and/or metadata information (e.g., from LIS 13, including for example, date of biopsy, patient medical record, stain type). For example, as elaborated herein, once a pathologist selects a case or sample 10 to work on, workflow manager 70 may guide the pathologist 15 through the diagnostic process by showing him or her (e.g., via HMI 30) relevant screens and information, step by step.

According to some embodiments, as elaborated herein, workflow manager module 70 may be configured to produce at least one workflow data element 70A that may include an assignment of a pathology case to one or more pathologists 15 of a plurality of pathologists in a pathology institute, based on each pathologist's 15 expertise, experience and/or workload.

In another example, workflow manager module 70 may be configured to produce at least one workflow data element 70A that may direct or enable one or more specific pathologists 15 to access (e.g., via workflow interface 330 of HMS 30) one or more data elements pertaining to an assigned case, including for example digitally scanned slides 25, information originating from LIS 13 (e.g., information regarding a patient's medical history and/or records), and/or data or metadata elements describing one or more aspects of one or more slides 25 (e.g., a date of biopsy, a stain type, etc.)

In another example, workflow manager module 70 may be configured to produce at least one workflow data element 70A. Workflow manager module 70 may then control the process or workflow of diagnosis according to workflow data element 70A, by “walking” or guiding the pathologist 15 through the diagnostic process. For example, workflow manager module 70 may communicate workflow data element 70A to workflow interface module 330 of HMI 30. Workflow interface module 330 may, in turn configure HMI 30 to show (e.g., on a pathologist's screen or via slide viewer 310) relevant information according to the workflow data element 70A. Such information may include, for example, relevant ROIs 41B and/or slides 25, LIS 13 related information (e.g., one or more data elements from previous medical examinations), information pertaining to classifier module 90 (e.g., ROI classification 91, slide classification 92, case classification 93), and the like.

According to some embodiments of the invention, workflow manager module 70 may be adapted to receive (e.g., from LIS 13, from classifier module 90, from IA module 40, etc.), one or more data elements pertaining to one or more digitally scanned pathology slides 25 of one or more pathology cases. The one or more data elements may be for example: (a) data elements originating from LIS 13, describing or corresponding to a pathology case; (b) data elements from IA, including one or more IA results (e.g., elements 41, 42 of FIG. 3); and (c) data elements of classifier module 90 (e.g., ROI classification 91, slide classification 92 and/or case classification 93).

According to some embodiments, workflow manager module 70 may produce a workflow data element 70A that may be or may include an ordered list of examination of the plurality of digitally scanned pathology slides 25 based on one or more ordering criteria. The one or more ordering criteria may include, for example: a classification of at least one ROI 91, a classification of at least one scanned pathology slide 92, a classification of at least one case 93 (e.g., provided by classifier module 90, so as to order the list according to a severity classification of ROIs, slides and/or cases); an extracted clinical feature 41 of the at least one scanned pathology slide, provided by IA 40 (e.g., assigning a high priority to cases that exhibit or include specific IA results 41, such as a specific diagnosis 41C, grade 41D, stage 41E, etc.); a profile of at least expert pathologist, provided by LIS 13 (e.g., an expertise of a specific pathologist 15); and medical data pertaining to the case, such as previous medical results and/or physician reports, provided by LIS 13. Workflow manager module 70 may subsequently collaborate with HMI 30, so as to present at least one digitally scanned pathology slide for examination via the HMI 30 (e.g., via slide viewer 310), based on the ordered list of examination (e.g., from a first slide 25 on the ordered list to a last slide 25 on the ordered list).

It may be appreciated that a tradeoff may exist between a diagnostic system's efficiency or throughput (e.g., the number of cases that may be processed or handled within a given timeframe) and accuracy (e.g., a portion of accurate or correct diagnoses from the overall number of processed cases). Theoretically, given a super-human pathologist 15 (e.g., a human expert who may not be tired), some embodiments may submit all slides 25 and all ROIs 41B to the pathologist's analysis, in order to obtain maximal accuracy. However, this approach may result in an inferior throughput. In a complementary manner, an approach that may include assigning all ROIs 41B and all slides 25 to automatic examination (e.g., classifying each case by classifier module 90, thus excluding a human pathologist 15 from the process of examination) may result in superior throughput, but may be limited in accuracy. In other words, the number of erroneous diagnoses may be dictated by the implementation and training of IA 40 and classifier module 90. This result may normally be unacceptable by a policy of a pathological institute.

According to some embodiments, workflow manager module 70 may collaborate with classifier module 90 to optimize the efficiency or throughput of diagnosis, in view of a given level of required accuracy.

In other words, some embodiments of the invention may include an improvement in technology to currently available systems of digital pathology, by applying a different workflow for each type of severity classification, and thus provide an optimal level of throughput to a process of pathological case examinations, while maintaining a predefined level of accuracy.

According to some embodiments, classifier module 90 may be configured to assign a severity score 90A to one or more elements such as ROIs 41B, slides 25 and/or cases, indicating a probability of malignancy. For example, a high severity score 90A may correspond to a high probability of malignancy of the relevant element and a low severity score 90A may correspond to a low probability of malignancy of the relevant element. In some embodiments, a severity score 90A of a slide 25 may correspond to (e.g., be equal to) a maximal severity score 90A of an ROI 41B included therein. Additionally, or alternatively, a severity score 90A of a pathology case may correspond to (e.g., be equal to) a maximal severity score 90A slides 25 pertaining to that case.

According to some embodiments, classifier module 90 may group or classify slides 25 (e.g., slide classification 92), pathological cases (e.g., case classification 93) and/or ROIs 41B (e.g., ROI classification 91) to groups or classifications of a set of severity classifications, according to the results 41 of IA module 40. The severity classifications may, for example, include or represent a “malignant” or “high priority” severity classification, for ROIs, slides and/or cases that are most likely cancerous; a “borderline” severity classification for borderline ROIs, slides and/or cases; and a “benign” severity classification for ROIs, slides and/or cases that are probably benign.

According to some embodiments, workflow manager module 70 may apply a different workflow for each classification so as to maximize the throughput and accuracy gains for that class.

For example, workflow manager module 70 may perform a triage of one or more pathological slides 10 and/or corresponding pathological cases, according to one or more IA clinical features 41, such as diagnosis data element 41C.

In another example, workflow manager module 70 may assign a case to a specific pathologist 15, based on the triage (e.g., assign specific cases according to expertise of each pathologist). Additionally, or alternatively, workflow manager module 70 may assign a case to a specific pathologist 15, based on the case severity classification 93. For example, workflow manager module 70 may assign “clear cut”, malignant cases to novice pathologists and assign borderline-severity cases (that may not be “clear-cut” decisions) to more experienced pathologists.

In another example, workflow manager module 70 may produce a workflow data element 70A that may be or may include an assignment of a priority for examination. For example, workflow manager module 70 may sort the scanned slides according to detected ROIs 41B and corresponding classifications (e.g., ROI classification 91) such that slides 25 and ROIs 41B of high severity may be assigned high priority (e.g., as part of workflow data element 70A) and may be presented to a pathologist 15 before slides 25 and ROIs 41B of lower severity. It may be appreciated that such assignment of priority may reduce turnaround time for severe (e.g., malignant) pathology cases.

In another example, workflow manager module 70 may produce a workflow data element 70A that may be or may include a property of representation of one or more ROIs 41B, slides 25 and/or cases 10, based on specific, respective classifications (e.g., 91, 92, 93). Workflow manager module 70 may then cooperate with HMI 30 so as to represent each classification differently on HMI 30. For example, elements (e.g., ROIs 41B, slides 25, cases) may be presented by corresponding or dedicated representation in HMI 30. Such dedicated representations may include, for example, specific colors in a heatmap representation, an outline of one or more relevant ROIs 41B in a viewed portion of a digitally scanned slide 25, etc. The dedicated representations may be overlaid (e.g., presented as a separate layer) on a currently viewed portion of a slide 25 in slide viewer 310.

Pertaining to the same example, as known in the art, it may be important to include (e.g., in report 150A) premalignant findings such as hyperplasia and/or inflammation. This may be particularly relevant in conditions of benign cases. According to some embodiments, workflow data element 70A may be, or may include a configuration of a gallery of relevant ROIs and/or slides, depending on the context. For example, in a condition of a slide that may be classified 92 as benign, it may be important to report occurrence of inflammation in the relevant sample or case 10, but it may not be important to indicate specific manifestations of inflammation in each slide 25. Therefore, workflow data element 70A may be or may include a gallery data element that may indicate tissue samples and/or locations, spanning multiple slides and showing the most likely areas to exhibit inflammation. In a similar manner, workflow manager 70 may be configured to report one or more pre-malignant feature (e.g., hyperplasia) per each slide. Workflow manager 70 may subsequently produce a workflow data element 70A that may be or may include a gallery data element, that may show tissue areas that are the most likely to include the pre-malignant feature (e.g., hyperplasia) in each slide. According to some embodiments, pathologist 15 may then review specific (e.g., isolated) areas that may be included in the gallery of workflow data element 70A and may decide whether to confirm the existence of the respective premalignant finding. Additionally, or alternatively, workflow manager 70 may collaborate with report interface 320 to automatically, in real-time, reflect the pathologist's confirmation of a premalignant finding in report 150A.

It may be appreciated that embodiments of the invention may provide an improvement over currently available systems for digital pathology by annihilating a need of the pathologist to perform context-switching between different types of diagnoses and reports (e.g., borderline ROIs and/or pre-malignant findings) pertaining to a pathology case. Moreover, it may be appreciated that embodiments of the invention may reduce the time required by a pathologist 15 for actively searching for pre-malignant findings.

Additionally, workflow data element 70A may be, or may include a gallery of relevant ROIs 41B and/or locations of pre-malignant findings. Workflow data element 70A may enable pathologist 15 to perform in-depth review of ROIs 41B included in, or referred by the gallery data element by, for example, enabling pathologist 15 to select (e.g., click on) specific icons that may be included in the gallery of workflow data element 70A, and consequently present the relevant region on slide viewer 310, in which all capabilities (e.g., zoom, pan, heatmap overlay) may be available.

In another example, workflow manager module 70 may cooperate with report interface 320 to present each element (e.g., ROIs 41B, slides 25, cases) to be presented on HMI 30 adjacent to specific report fields, relevant to the elements' respective severity class.

According to some embodiments, IDS system 100 may include an ML-based preordering module 160. Preordering module 160 may receive at least one of: an extracted clinical feature 41, a classification of a digitally scanned pathology slide 92, a classification of a pathology case 93 and/or an indication of slide quality (e.g., included in slide-related data element 42B, as elaborated herein). Preordering module 160 may be trained (e.g., by receiving supervisory information from a human pathologist) to produce (e.g., predict, as commonly referred to in the art) at least one preorder suggestion data element 160A that may include a recommendation for preparation of a number N(SLIDES) of digitally scanned pathology slides. For example, given an initial number of digitally scanned slides 25 pertaining to a specific pathology case, preordering module 160 may be trained to determine a quantity of additional slides that should be ordered, and specific properties of the additional slides.

Additionally, or alternatively, ML-based preordering module 160 may be trained to produce a preorder suggestion data element 160A that may include a number of N(SLIDES) of slides, so as to optimize throughput and/or TAT of the process of pathology examination, while maintaining a predefined level of accuracy. It may be appreciated that the type and/or properties of the preordered slides may be dependent upon the findings of IA 40 (e.g., dependent upon clinical features 41 and/or geometric features 42), as elaborated in the examples that follow.

Additionally, or alternatively, ML-based preordering module 160 may be trained to produce a minimal number N_{(SLIDES)} of slides so as to optimize throughput and/or TAT of the process of pathology examination, while adhering to a policy or a set of rules and/or restrictions of the pathology institute. For example, ML-based preordering module 160 may be restricted to preorder a limited number N_{(SLIDES)} of slides, according to a budget limitation. ML-based preordering module 160 may thus be adapted to produce specific preorder suggestions 160A that may produce the required information and may adhere to the budget limitation.

Workflow manager module 70 may receive at least one recommendation for preparation of a number N_{(SLIDES)} of digitally scanned pathology slides and may cooperate with sample processing module 20 to preorder at least one slide and/or performance of at least one process of processing module 20 (e.g., a slice 20A, a stain 20B and/or a scan 20C).

It may be appreciated that automatically (or semi-automatically) ordering of slides as done by embodiments of the present invention may provide a dual improvement over currently available systems for examination of pathology slides.

In a first aspect, automatic preordering of slides may improve a turnaround time for one or more specific slides or slide types, since the pathologist may have the slides ready and may only need to examine the respective case once (e.g., rather than performing a first examination, ordering additional required slides, and then returning to the case to finalize the examination).

In a second aspect, the reduction of TAT as elaborated above may also improve the global efficiency or throughput (e.g., a number of cases that may be examined during a predefined period of time) of the pathology institute. For example, it may be appreciated that examination of a pathology case more than once (e.g., first time diagnosing and ordering slides, second time re-diagnosing using the newly ordered slides) may consume unnecessary time and effort by a pathologist. The pathologist may need to re-enter the case (in an LIS system 13, in the slide viewer 310, etc.), recall what they had previously found, re-examine slides and/or ROIs that have already been viewed, etc. Such “context switching” may waste a pathologist's time and effort, and may decrease the pathologist's overall throughput. It may be appreciated that some embodiments of the invention may include an improvement over currently available systems for digital pathology examination by having all the required data ready for the pathologist at a first read, avoiding waste of time and effort by the pathologist, and increasing an overall throughput of the pathologist.

For example, as elaborated herein, slide-related data element 42B may include an indication of properties of quality of the digitally scanned slide 25. In a condition that a pathology sample 10 has been processed such that the subsequent digitally scanned slide 25 is impaired or of poor quality, IA 40 may identify the impaired condition of digitally scanned slide 25. For example, IA 40 may identify a condition in which a digitally scanned slide 25 is improperly focused, improperly stained, contains an unusual amount of tissue (e.g., insufficient or abundant), contains poorly cut tissue fragments, and the like and slide-related data element 42B may include an indication of such impairment.

Preordering module 160 may subsequently produce a recommendation for preparation of a number of zero or more (e.g., N_{(SLIDES)}) digitally scanned pathology slides 25 so as to remedy the impairment. For example, preordering module 160 may produce a recommendation to re-stain a slide 25, re-scan a slide 25 and/or produce an entirely new slide 25, including new slices of sample 10.

Workflow manager module 70 may subsequently produce or emit a notification or a message to sample processing module 20 so as to remedy the impairment according to the recommended action of preordering module 160 (e.g., produce a new slice, re-stain a slice, re-scan a slice, etc.). Additionally, or alternatively, workflow manager module 70 may produce or emit a notification (e.g., an email notification to a user via HMI 30 or via output device 8 of FIG. 2) to a user (e.g., pathologist 15) notifying them of the impairment.

In another example, ML module 71 may be trained (e.g., by receiving supervisory information from a human pathologist) to learn whether and/or which processes should be performed by processing module 20, given specific clinical features. For example, in a condition that diagnosis data element 41C includes information pertaining to, or representing a specific clinical condition (e.g., a specific type of suspected malignancy), ML module 71 may identify that clinical condition as one that requires a specific process (e.g., a specific type of staining 20B) by processing module 20. Workflow manager module 70 may subsequently produce or emit a notification or a message (e.g., to a user, via HMI 30, via output device 8 of FIG. 2 and/or directly to sample processing module 20), so as to perform the required process (e.g., produce a digitally scanned slide 25 having the required staining).

For example, it may be appreciated that a pathology expert may require a determination of the existence of Helicobacter Pylori (HP) in gastric biopsies. As known in the art, in certain conditions HP may be easily detectable in H&E slides, and so H&E slides may suffice for that purpose. In other conditions, HP may not be easily detectable in H&E slides, although a physician may suspect HP does exist in the examined tissue (e.g., due to apparent inflammation). In conditions such as the latter, a pathologist may require an additional slide 25 that may include a specific stain (e.g., Geimsa or IHC). Some embodiments of the invention (e.g., preorder module 160A) may include an improvement over currently available systems of digital pathology by ordering the required stain prior to inception of the examination process by the pathologist, and thus saving time, reducing TAT and increasing efficiency of the pathology institute.

As elaborated herein, workflow manager module 70 may collaborate with classifier module 90 to optimize the throughput of diagnosis, in view of a given level of required accuracy. According to some embodiments, IDS 100 may include an ML-based optimization module 50.

Optimization module 50 may receive at least one digital scan 25 of a pathology slide that may have a low-risk (e.g., benign) severity slide classification 92. In other words, optimization module 50 may receive a slide 25 that has been assigned (e.g., by classification module 90) a low severity score 90A and/or has been attributed (e.g., by classification module 90) a “low-risk” or “benign” slide classification 92.

According to some embodiments, optimization module 50 may receive (e.g., from IA 40) at least one ROI 41B data element that may be included in the received low-risk slide 25 (and hence may be classified as “low-risk” or “benign”). Additionally, optimization module 50 may receive a severity score 90A, corresponding to the represented “low-risk” or “benign” ROI 41B.

According to some embodiments, optimization module 50 may be adapted to select an optimized number N_{(POI)} of the highest-scored (e.g., having the highest severity scores) ROIs 41B in the “low risk” scanned pathology slide 25. The selected N_{(POI)} highest-scored ROIs in the “low risk” scanned pathology slide 25 may hereinafter be referred to as Points of Interest (POIs) 41B′. The term “optimized” may refer herein to the number N_{(POI)} of selected POIs 41B′ in a sense that the ML-based optimization module 50 may be trained to select a minimal number N_{(POI)} of POIs, so as to optimize a throughput of the process of pathology examination while maintaining a predefined level of accuracy, as elaborated herein.

According to some embodiments, workflow manager 70 may subsequently produce a recommendation (e.g., via HMI 30) to examine (e.g., on slide viewer 310) the selected N_{(POI)} POIs of the scanned pathology slide 25. For example, workflow manager 70 may communicate at least one data element pertaining to the N_{(POI)} POIs 41B′ of the scanned pathology slide 25 to HMI 30. HMI 30 may, in turn display a gallery of the N_{(POI)} POIs 41B′ in the slide, each at an appropriate magnification, to enable a pathologist 15 to quickly examine the POIs 41B′ and decide whether a further review of the slide is required.

According to some embodiments, optimization module 50 may be trained to predict the optimal number N_{(POI)} of POIs during at least one of an initial stage and a training stage.

For example, during an initial stage, a milestone level of accuracy of a pathology examination by one or more (e.g., specific) pathologists of a pathology institute may be calculated in the absence of IDS system 100, as a ratio of correct diagnoses from the overall number of examined pathological cases. Additionally, a milestone level of throughput of pathology examination by one or more (e.g., specific) pathologists of the pathological institute may be calculated in the absence of IDS system 100 as the overall number of examined pathological cases during a predefined period (e.g., a week).

As explained above, a tradeoff may exist between accuracy and throughput of a process of examination. It may be appreciated by a person skilled in the art that, due to such tradeoff, a small value (e.g., 0) of N_{(POI)} may increase the throughput of examination (e.g., by pathologists ignoring “low-risk” or “benign” slides 25 altogether) but compromise the accuracy of examination (e.g., by letting malignant cases go unexamined). In a complementary manner, a large value of N_{(POI)} (e.g., selecting all the POIs 41B′ of the “low-risk” or “benign” slides 25) may result in a high level of accuracy (not taking into account the effect of fatigue on pathologists) but may decrease the throughput of examination (e.g., by requiring pathologists to examine each POI 41B′ in “low-risk” or “benign” slides).

During a training stage, optimization module 50 may be trained so as to predict an optimal value N_{(POI)} of POIs 41B′, so as to increase the throughput of the pathological examination (e.g., of a specific pathologist) beyond a predefined level (e.g., the milestone level) of throughput, while maintaining at least a predefined level (the calculated milestone level) of accuracy of the pathological examination (e.g., of the specific pathologist).

It may be appreciated by a person skilled in the art that optimization module 50 may be further configured to select or predict (e.g., in an iterative process) a value of N_{(POI)} that may exploit the aforementioned tradeoff. Thus, optimization module 50 may produce a value of N_{(POI)} POIs 41B′, so as to obtain a level of accuracy that may be improved (e.g., higher) in relation to the milestone accuracy level, while maintaining an throughput level that is also improved (e.g., higher) in relation to the milestone throughput level.

As known in the art, ML-based optimization module 50 may be trained to predict or produce a value N_{(POI)} of POIs 41B′ based on an optimization of a predefined target function. For example, the target function may include optimizing a number N_{(POI)} that may be equal to or smaller than a pre-defined number N of POIs per slide, such that the probability of missing a malignant case (e.g., the probability that the slide contains cancer, but the cancer isn't in one of the predefined number of N POIs) is minimal In another example, a target function may include setting a desired upper bound on the probability to miss cancer (e.g., 0.1% or 0.01%) and then identifying the smallest number of POIs per slide that may guarantee such level of accuracy. It may be appreciated that other target functions that may combine efficiency (e.g., the average number of POIs per slide) and accuracy (e.g., the expected probability that a malignancy is included within at least one POI in a slide) may also be possible.

Some embodiments of the invention may present the one or more N_{(POI)} POIs 41B′ to the pathologist (e.g., as flags, circles and/or other markings on the slide viewer 310) and may prompt or guide the pathologist to examine each of the N_{(POI)} POIs 41B′. It may be appreciated that, if all N_{(POI)} POIs 41B′ are found benign, the pathologist 15 may diagnose (e.g., produce a report 150A) the entire slide and/or case as benign, knowing that the probability that a malignancy is included therein may be slim.

According to some embodiments, the training of optimization module 50 may be specific to individual pathologists (e.g., producing a different predicted value of N_{(POI)} for each pathologist). Additionally, or alternatively, the training of optimization module 50 may be specific to diagnoses (e.g., producing a different predicted value of N_{(POI)} for each type of diagnosis 41C). Additionally, or alternatively, the training of optimization module 50 may be specific to at least one property of the working environment (e.g., producing a different predicted value of N_{(POI)} for different times of the day).

It may be appreciated by a person skilled in the art that some embodiments of the invention may provide an improvement over currently available methods of pathology diagnosis, by (a) classifying the slides according to severity classifications 92; and (b) facilitating an adaptive selection of the number of ROIs 41B in the “benign” classification, that are required for examination. Thus, instead of the two extremes, one being automatic screening (e.g., filtering out) of ROIs 41B and the other being a full manual review, some embodiments may enable pathologists to examine a set of locations or ROIs that may be identified by the algorithm in each slide and that may be identified as being of a low severity risk. This workflow may at least maintain the required level of accuracy (e.g., the milestone accuracy), and increase throughput of the examination, as the pathologist may be enabled to skip through or quickly review ROIs of the slide.

As elaborated herein, classifier module 90 may classify one or more ROIs 41B, slides 25 and/or cases 10 to one of a plurality of severity classifications (e.g., malignant, borderline and benign), based on a severity score 90A. It may be appreciated that the borderline classification may allow some embodiments of the invention (e.g., workflow manager 70) to define a separate or fine-tuned workflow for such classified elements (e.g., ROIs 41B, slides 25 and/or cases 10 of borderline classification).

For example, workflow manager 70 may collaborate with preorder module 160 to define a specific policy for pre-ordering of slides and/or stains (e.g., so as to enhance a human pathologist's capability of diagnosing the examined case).

In another example, as elaborated herein, workflow manager 70 may assign borderline elements (e.g., ROIs 41B, slides 25 and/or cases 10 of borderline classification) to specific (e.g., highly experienced) pathologists.

In another example, as elaborated herein, workflow manager 70 may assign a specific priority to examination of borderline cases. For example, borderline cases may be assigned a high priority as they tend to be complicated, as may be appreciated by a person skilled in the art.

In yet another example, workflow manager 70 may communicate with workflow interface 330 of HMI 30. Workflow interface 330 may subsequently assign specific viewing properties to display (e.g., by slide viewer 310) one or more portions of digitally scanned slides 25. For example, in a condition of a borderline severity slide, workflow interface 330 may configure slide viewer 310 of HMI 30 to present the one or more portions with a neutral indication (e.g., show one or more ROIs 41B without any highlight, with or without indication of a respective severity score), so as not to bias pathologist's decision 15 by presenting indecisive classification (e.g., 91, 92, 93) results.

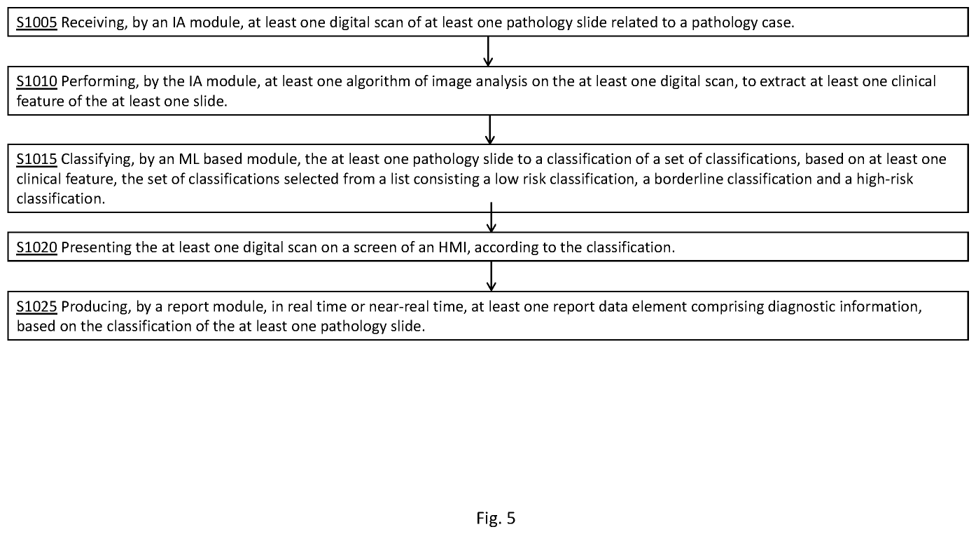

Reference is now made to FIG. 5, which is a flow diagram, depicting a method of managing workflow of examination of pathology slides by one or more processors (e.g., element 2 of FIG. 2), according to some embodiments.

As shown in step S1005, an IA module (e.g., element 40 of FIG. 3) may receive at least one data element including a digital scan (e.g., element 25 of FIG. 3) of at least one pathology slide related to a pathology case (e.g., element 10 of FIG. 3).

As shown in step S1010, IA module 40 may performing, at least one algorithm of image analysis on the at least one digital scan, to extract at least one IA result such as a clinical feature (e.g., element 41 of FIG. 3) of the at least one digitally scanned slide 25.

As shown in step S1015, an ML based module (e.g., element 90 of FIG. 4) may classify the at least one pathology slide 25 to a classification of a set of classifications, based on at least one clinical feature, as elaborated herein (e.g., in relation to FIG. 4). According to some embodiments, the set of classifications may be or may include, for example, a low risk classification, a borderline classification and a high risk classification.

As shown in step S1020, embodiments of the invention may include presenting the at least one digitally scanned slide 25 on a screen of an HMI (e.g., element 30 of FIG. 4), according to the classification (e.g., as elaborated herein, in relation to FIG. 4).

As shown in step S1025, embodiments of the invention may include producing, by a report module (e.g., element 150 of FIG. 4), in real time or near-real time, at least one report data element comprising diagnostic information, based on the classification of the at least one pathology slide (e.g., as elaborated herein, in relation to FIG. 4).

As elaborated herein, some embodiments of the present invention provide a practical application for management of a pathology examination process by an expert pathologist (e.g., in a pathology institute). Some embodiments of the invention include employment of automated tools that introduce a plurality of improvements over currently available technology for digital pathology, as elaborated above. Such improvements ultimately amount to: (a) decreasing turnaround time for examination of slides, (b) increasing throughput of the examination process, and (c) increasing accuracy and decreasing the error rate of the examination process.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A system for managing a process of pathology examination, the system comprising:
an image-analysis (IA) module, configured to receive at least one digital scan of at least one pathology slide related to a pathology case, and perform at least one algorithm of image analysis thereon to extract at least one clinical feature of the at least one slide;
a human-machine interface (HMI), adapted to present the at least one digital scan on a screen;
a first machine-learning (ML) based module, adapted to classify the at least one pathology slide to a classification of a set of classifications based on at least one clinical feature, the set of classifications selected from a list consisting a low risk classification and a high risk classification; and
a report module, adapted to produce in real time, at least one report data element comprising diagnostic information, based on the classification of the at least one pathology slide.

2. The system according to claim 1, wherein the at least one extracted clinical feature corresponds to one or more regions of interest (ROIs) of the at least one slide, and wherein the first ML-based module is adapted to classify the ROIs to a classification of the set of classifications based on the at least one extracted clinical feature, and wherein classifying the at least one pathology slide is done based on classification of the one or more ROIs of the at least one pathology slide.

3. The system according to claim 1, wherein the first MU-based module is adapted to classify pathology case to a classification of the set of classifications, based on the at least one at least one classifications of a pathology slide related to the pathology case.

4. The system of claim 1 wherein the HMI is configured to receive, from a user, at least one viewing context data element, and present the digitally scanned pathology slide according to the at least one viewing context data element, in conjunction with the at least one diagnostic report data element on the screen.

5. The system according to claim 1 wherein the at least one viewing context data element is selected from a list consisting of a selection of a pathology case, a selection of a specific digitally scanned pathology slide, a selection of one or more ROIs, a selection of panning, a selection of zoom, a selection of brightness and a selection of contrast.

6. The system according to claim 1, wherein the HMI is configured to produce a viewing suggestion according to at least one of: the at least one extracted clinical feature, a classification of at least one ROI and a classification of at least one pathology slide.

7. The system according to claim 1, wherein the HMI is further configured to receive, from a user, at least one expert data element pertaining to the presented at least one pathology slide, and wherein the report module is further configured to integrate the at least one expert data element into the at least one report data element to produce an integrated report data element.

8. The system according to claim 1, wherein the HMI is further configured to present the integrated report data element on the screen according to the at least one viewing context data element.

9. The system according to claim 1, further comprising a quality control module, configured to:
receive, from a lab information sub-system (LIS) at least one LIS data element pertaining to the pathology case; and
analyze the integrated report data element in view of the at east one LIS data element to detect at least one discrepancy,
wherein the HMI is further configured to present a notification of the detected at least one discrepancy on the screen.

10. The system of claim 1, further comprising an ML-based optimization module, configured to:
receive at least one digital scan of a pathology slide of the low risk classification;
select a number N_{(POI)} of ROIs of low risk classification, of the least one received digitally scanned pathology slide; and
produce a recommendation to examine the N_{(POI)} ROIs of the least one received digitally scanned pathology slide,
wherein the ML-based optimization module is trained to select a minimal number N_{(POI)} of ROIs, so as to optimize a throughput of the process of pathology examination, while maintaining a predefined level of accuracy of the process of pathology examination.

11. The system according to claim 1, further comprising a workflow manager module, adapted to:
produce an ordered list of examination of the plurality of digitally scanned pathology slides based on one or more ordering criteria; and
present at least one digitally scanned pathology slide for examination via the HMI based on the ordered list of examination,
wherein the one or more ordering criteria is selected from a list consisting of: a classification of at least one pathology case, a classification of at least one scanned pathology slide, a classification of at least one ROI, at least one extracted clinical feature, a profile of at least one expert pathologist, and at least one LIS data element pertaining to the pathology case.

12. The system according to claim 1, wherein the set of classifications further comprises a borderline risk classification.

13. The system of claim 1 further comprising an ML-based preordering module, configured to:
receive at least one of a clinical feature and a classification of a digitally scanned pathology slide; and
produce at least one recommendation for preparation of a number N_{(SLIDES)} of digitally, scanned pathology slides,
wherein the ML-based preordering module is trained to produce a minimal number N_{(SLIDES)} of slides, so as to optimize a throughput of the process of pathology examination while maintaining a predefined level of accuracy.

14. The system of claim 1, wherein the IA module is further configured to extract at least one tissue-related property pertaining to one or more tissue fragments of the at least one pathology slide, wherein the tissue-related property is selected from a list comprising: a length of a tissue fragment, a shape of a tissue fragment, an area of a tissue fragment, a volume of a fragment and a number of tissue fragments in the scanned pathology slide.

15. The system of claim 14, further comprising a quality control module, configured to analyze at least one tissue-related property of a tissue fragment pertaining to a specific pathology case, to determine a discrepancy between metadata of one or more scanned pathology slides of the specific pathology case, and the tissue-related property.

16. The system according to claim 1, wherein the report module is further configured to integrate at least one tissue-related property of a tissue fragment pertaining to a specific pathology case into the at least one report data element, to produce an integrated report data element.

17. The system of claim 1, wherein the at least one clinical feature is selected from a list consisting: a high priority medical condition, a secondary medical condition, one or more ROIs corresponding to the high priority medical condition, one or more ROIs corresponding to the secondary medical condition, a diagnosis of the high priority medical condition, a diagnosis of the secondary medical condition, a grade of the high priority medical condition, a stage of the high priority medical condition, a morphologic property of the high priority medical condition, a morphologic property of the secondary medical condition, a cytologic property of the high priority medical condition and a cytologic property of the secondary medical condition.

18. The system of claim 1, wherein the IA module is further configured to extract at least one slide-related properly pertaining to the scanned pathology slide, and wherein the slide-related property is selected from a list consisting of: a location of an extracted clinical feature within the slide, a quantization of the extracted clinical feature within the slide, a dimension of a tumor across one or more digitally scanned pathology slides, a percentage of a tumor across one or more digitally scanned pathology slides, a mitoses count, a percentage per grade, an IHC quantification, an indication of a distance between cancerous cells and a border of a tissue, and an indication of whether cancerous cells have metastasized beyond the predefined border.

19. The system according to claim 1, wherein the report module is further configured to integrate at least one slide-related property of a slide pertaining to a specific pathology case into the at least one report data element, to produce an integrated report data element.

20. The system of claim 1 wherein the IA module is further configured to:
receive one or more data elements selected from a list consisting: (a) an expert pathologist data element pertaining to diagnosis of a presented pathology slide and (h) a pathology report data element; and
use the received one or more data elements as supervisory data, to further train and improve the extraction of the at least one clinical feature.

21. A method of managing a process of pathology examination, the method comprising:
receiving at least one digital scan of at least one pathology slide related to a pathology case, and performing at least one algorithm of image analysis thereon to extract at least one clinical feature of the at least one slide;
classifying at least one ROT of the at least one digital scan of at least one pathology slid e to a classification of a set of classifications, based on at least one clinical feature;
producing a workflow data element based on the classification of at least one ROI; and
controlling the process of diagnosis, based on the at least one workflow data element, by guiding a pathologist through the diagnostic process on a human-machine interface.

22. A method of managing a process of pathology examination, the method comprising:
receiving, by an IA module, at east one digital scan of at least one pathology slide related to a pathology case;
performing, by the IA module, at least one algorithm of image analysis on the at least one digital scan, to extract at least one clinical feature of the at least one slide;
classifying, by an ML based module, the at least one pathology slide to a classification of a set of classifications, based on at least one clinical feature, the set of classifications selected from a list consisting a low risk classification and a high risk classification
presenting the at least one digital scan on a screen of an HMI, according to classification; and
producing, by a report module, in real time, at least one report data element comprising diagnostic information, based on the classification of the at least one pathology slide.
